(19) United States
(12) Patent Application Publication
(10) Pub. No.: US 2026/0358357 A1
(43) Pub. Date: Nov. 12, 2026

(54) Qualitative Test For Direct Oral Anticoagulants (DOACS)

(71) Applicant: ROCHE DIAGNOSTICS OPERATIONS, INC., Indianapolis, IN (US)

(72) Inventor: Sascha MEYER DOS SANTOS, Portimão (PT)

(21) Appl. No.: 19/715,574

(22) Filed: Jun. 22, 2026

Related U.S. Application Data
(63) Continuation of application No. PCT/EP2024/087250, filed on Dec. 18, 2024.

(30) Foreign Application Priority Data
Dec. 19, 2023 (EP) 23218227.9

(51) Int. Cl. G01N 33/86; C12Q 1/00; C12Q 1/56; G01N 27/327

(52) U.S. Cl. CPC G01N 33/86; C12Q 1/005; C12Q 1/56; G01N 27/3271; G01N 27/3276; G01N 2333/96433; G01N 2333/96444; G01N 2333/96486

(57) ABSTRACT

The present disclosure provides methods, kits and devices for determining an anticoagulant in a blood sample. The method may include one or more of the following steps: a) providing a composition comprising: i) thrombin or a prothrombin activator converting prothrombin into thrombin that is factor Xa (FXa) inhibitor insensitive, or mixtures thereof; and ii) FXa or a prothrombin activator converting prothrombin into thrombin that is FXa inhibitor sensitive, or mixtures thereof; b) contacting a blood sample with the composition thereby generating a mixture of the composition with the blood sample, wherein the mixture comprises at least 0.01 nkat of thrombin activity and at least 0.05 nkat of FXa activity; c) measuring thrombin activity using a substrate capable of detecting thrombin activity; d) comparing the measured thrombin activity to a reference; and e) determining the anticoagulant based on the comparison.

### CROSS-REFERENCE TO RELATED APPLICATIONS

This application is a continuation of International Patent Application No. PCT/EP2024/087250, filed Dec. 18, 2024, which claims the benefit of priority to European Patent Application No. 23218227.9, filed Dec. 19, 2023, the disclosures of which are herein incorporated by reference in their entirety.

### TECHNICAL FIELD

The present disclosure relates to the field of point-of-care diagnostics. In particular, it relates to methods, kits, and devices for detecting, or determining an amount or concentration of, an anticoagulant in a blood sample.

### BACKGROUND

Direct oral anticoagulants (DOACs) are active agents approved for multiple thromboembolic disorders and provide advantages over existing agents. As with all anticoagulants, management protocols for the eventuality of bleeding are important.

Although DOACs do not need routine coagulation monitoring for dose adjustment, there are instances when measurement of the drug anticoagulant effect may be useful. They include before initiation of treatment, before surgical or invasive procedures, on the occasion of hemorrhagic or thrombotic events, and whenever immediate reversal of anticoagulation is needed or the coagulation capacity of a subject needs to be known. If patients experience bleeding or need procedural interventions, laboratory monitoring still has to be performed.

A number of laboratory tests developed for understanding the coagulation cascade and for determining coagulation capacity in a blood sample relate to the determination of the clotting time. These include for example prothrombin time, activated partial thromboplastin time, activated clotting time and thrombin time tests (Harris et al. 2013, Trends in Analytical Chemistry 50: 85-95). Recent detection methodologies for these tests include chromogenic, immunologic, turbidimetric, photo-optical and mechanical detection. While coagulation analysis generally has been automated and several point-of-care devices are commercially available, reliably determining the presence of DOACs in a blood sample is still a challenge. The number of readily available tests to detect and monitor these agents is still very limited.

U.S. Patent Application Pub. No. 2016/0032355A1 decribes a method for detecting the presence of a direct oral anticoagulant in a patient's sample. The method comprises performing a clotting assay with a blood sample suspected to contain an anticoagulant in comparison to a control sample without anticoagulant. A clotting time exceeding that of the control sample indicates presence of an anticoagulant in the blood sample. The anticoagulant may be classified as a direct thrombin inhibitor or an anti-Factor Xa anticoagulant subsequently by performing a further separate clotting assay. The measurements are performed using thromboelastography and hence require specialized laboratory equipment. Hence, the method is rather laborious, time consuming and therefore cost intensive.

### BRIEF SUMMARY

The following passages introduce a selection of concepts in a simplified form that are further described below in the Detailed Description. This summary of selected aspects is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In view of the disadvantages of current solutions, there remains an unmet need in the art for a simple and rapid method to test for the presence of DOACs in a blood sample, and in particular, for a one-step method that is compatible with point-of care diagnostics. Furthermore, there is a need for determining the presence of all types of currently available DOACs, i.e., factor IIa inhibitor type anticoagulants and factor Xa inhibitor type anticoagulants. The methods, working electrodes, devices, analyte sensors, systems, and a kits described herein address these needs, in addition to providing various other benefits as shall become apparent in view of the present disclosure.

In some aspects, the present disclosure relates to a method for determining an anticoagulant in a blood sample, comprising the following steps: a) providing a composition comprising: i) thrombin or a prothrombin activator converting prothrombin into thrombin that is factor Xa (FXa) inhibitor insensitive, or mixtures thereof; and; ii) FXa or a prothrombin activator converting prothrombin into thrombin that is FXa inhibitor sensitive, or mixtures thereof; b) contacting a blood sample with the composition thereby generating a mixture of the composition with the blood sample, wherein the mixture comprises at least 0.01 nkat of thrombin activity and at least 0.05 nkat of FXa activity; c) measuring thrombin activity using a substrate capable of detecting thrombin activity; d) comparing the measured thrombin activity to a reference; and e) determining the anticoagulant based on the comparison.

The present methods are advantageous in that they provide a simple, fast and reliable way of determining anticoagulants, in particular DOACs, in a blood sample. Furthermore, such methods are suitable for use in point-of-care devices, and allow one to rapidly and reliably assess whether relevant amounts of DOACs are present in a patient's blood sample. In doing so, such methods provide a rapid test for determining whether the coagulation status of a patient is safe for intervention (surgery, stroke thrombolysis, etc.) or not. Determining the DOAC anticoagulation status before intervention can reduce bleeding risks and prevent potentially adverse blood coagulation affecting treatments and help the treating healthcare professional to choose the best treatment option. Uncontrolled bleeding due to DOACs pharmacodynamic effects in patients under interventions can easily become life-threatening and clinically complex situations. Moreover, the methods described herein may additionally be used to discriminate between different types of DOACs, namely factor IIa inhibitor type anticoagulants and factor Xa inhibitor type anticoagulants, in a one-step reaction. Additional features and inventive aspects of the methods, devices, kits, and other aspects of the disclosure shall become apparent in view of the non-limiting examples described herein.

### BRIEF DESCRIPTION OF THE DRAWINGS

Various features and advantages of aspects of the present disclosure will be more fully understood from the following detailed description of illustrative embodiments taken in conjunction with the accompanying drawings in which:

FIG. 1 provides an overview of the clotting cascade and a detection mechanism implemented by certain methods disclosed herein.

FIG. 2 shows a plot of the internal clotting time (ICT) by amperometric determination.

FIG. 3 shows the dose-response curves of the internal clotting time (ICT) versus the concentration of DOAC in a sample. The activities of the activators in the composition were chosen in a way that at 30 ng/mL the ICT for the different DOACs is essentially identical.

### DETAILED DESCRIPTION

The present disclosure relates to methods, assays, kits and devices for determining the presence of anticoagulants (e.g., DOACs, such as factor IIa inhibitor type anticoagulants and factor Xa inhibitor type anticoagulants) in a sample. The inventive embodiments described herein enable accurate and easily deployable point-of-care devices to detect and/or measure such anticoagulants in a clinical setting, promoting improved patient care and therapeutic outcomes.

Prior to describing the various embodiments, the following definitions are provided and should be used unless otherwise indicated.

It is to be understood that as used in the specification and in the claims, “a” or “an” can mean one or more, depending upon the context in which it is used. Thus, for example, reference to “an” item cell” can mean that at least one item can be utilized.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which a solely and exclusively consists of B) and to a situation in which, besides B, one or more further elements are present in entity A, such as element C, elements C and D or even further elements. The term “comprising” also encompasses embodiments where only the items referred to are present, i.e. it has a limiting meaning in the sense of “consisting of”, or not.

Further, as used in the following, the terms “particularly”, “more particularly”, “typically”, and “more typically”, “specifically” and “more specifically” or similar terms are used in conjunction with additional/alternative features, without restricting alternative possibilities. Thus, features introduced by these terms are additional/alternative features and are not intended to restrict the scope of the claims in any way. The inventive aspects of the present disclosure may, as the skilled person will recognize, be performed by using alternative features. Similarly, features introduced by “in an embodiment of the disclosure” or similar expressions are intended to be additional/alternative features, without any restriction regarding alternative embodiments of the inventive aspects described herein, without any restrictions regarding the scope of the claimed invention and without any restriction regarding the possibility of combining the features introduced in such way with other additional/alternative or non-additional/alternative features of the inventive aspects described and claimed herein.

Furthermore, it will be understood that the term “at least one” as used herein means that one or more of the items referred to following the term may be used in accordance with the present disclosure. For example, if the term indicates that at least one sampling unit shall be used this may be understood as one sampling unit or more than one sampling unit, i.e. two, three, four, five or any other number. Depending on the item the term refers to the skilled person understands as to what upper limit the term may refer, if any.

The term “about” as used herein means that with respect to any number recited after said term an interval accuracy exists within in which a technical effect can be achieved. Accordingly, about as referred to herein, preferably, refers to the precise numerical value or a range around said precise numerical value of ±20%, preferably ±15%, more preferably ±10%, or even more preferably ±5%.

Furthermore, the terms “first”, “second”, “third” or “(a)”, “(b)”, “(c)”, “(d)” etc. and the like in the description and in the claims are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein. In case the terms “first”, “second”, “third” or “(a)”, “(b)”, “(c)”, “(d)”, “i”, “ii” etc. relate to steps of a method or use or assay, there is no time or time interval coherence between the steps, i.e. the steps may be carried out simultaneously or there may be time intervals of seconds, minutes, hours, days, weeks, months or even years between such steps, unless other-wise indicated in the application as set forth herein above or below.

It is to be understood that this inventive aspects of the present disclosure are not limited to the particular methodology, protocols, reagents etc. described herein as these may vary. It is also to be understood that the terminology used herein is for the purpose of describing particular embodiments only, and is not intended to limit the scope of the claimed invention. Unless defined otherwise, all technical and scientific terms used herein have the same meanings as commonly understood by one of ordinary skill in the art.

The methods of the present disclosure are typically implemented as ex vivo methods. The methods described herein may comprise the steps mentioned before, i.e., they may consist of those steps or may comprise additional steps. Moreover, the method may, typically, be assisted by automation where feasible. For example, the application and handling of samples may be assisted by robotics, while the determination of the anticoagulant may be carried out by a data processing unit adapted for carrying out the necessary comparisons and/or calculations.

The term “determining” as used herein refers to qualitative and/or quantitative determination of an anticoagulant in a blood sample. The term, in particular, encompasses the determination of the presence or absence or the determination of the absolute or relative amount or the determination of the type of anticoagulant. The determination of the absolute of relative amounts of anticoagulant may refer to determining whether the amount of anticoagulant exceeds a certain threshold or not, or whether an anticoagulant is present in the sample within a certain concentration threshold. Specifically, the determination may further comprise discriminating between different inhibitor types of anticoagulants; more specifically discriminating between factor IIa inhibitor type anticoagulants and factor Xa inhibitor type anticoagulants.

Determining the anticoagulant as referred to in step e) of a method according to the present disclosure may be achieved by deducing or calculating the presence, absence or amount of anticoagulant from the measured thrombin activity. Further details are discussed elsewhere herein. Typically, the amount of thrombin formed in the test and the kinetics of thrombin accumulation after the blood sample has contacted the test mixture is dependent on the concentration of the DOAC and may further depend on the mode of action of the DOAC: DOACs inhibiting factor IIa may in particular have slightly different thrombin accumulating kinetics than DOACs inhibiting factor Xa, since factor X sits upstream of prothrombin in the waterfall blood coagulation cascade model. Therefore, specifically a kinetic analysis as to how thrombin activity develops in time is a key to discriminating between DOACs inhibiting factor IIa and DOACs inhibiting factor Xa. The drug classes are commonly considered to have their “fingerprint” in time series analysis.

The term “anticoagulant” refers to any active agent with anticoagulating properties known to the skilled artisan; anticoagulating properties are properties of the anticoagulant that result in and/or contribute to prevention or reduction of coagulation of blood, prolonging the clotting time. In particular, the term anticoagulant referred to by the present disclosure is an oral anticoagulant, in particular, a directly acting oral anticoagulant or direct oral anticoagulant (DOAC). The term “oral anticoagulant” refers to an anticoagulating substance that may be administered orally; “directly acting oral anticoagulants” are considered as oral anticoagulants having a rapid onset of action and relatively short half-lives. The anticoagulant of the present disclosure typically is a direct oral anticoagulant of the factor IIa (thrombin) inhibitor type or of the FXa inhibitor type. Direct oral anticoagulants of the factor IIa (thrombin) inhibitor type include, e.g., dabigatran, ximelagatran, atecegatran metoxil (AZD0837). Direct oral anticoagulant of the factor FXa inhibitor type include, e.g., rivaroxaban, edoxaban, betrixaban, apixaban, letaxaban (TAK-422), eribaxaban, and darexaban. In some aspects, the anticoagulant is a DOAC selected from the group consisting of: dabigatran, rivaroxaban, edoxaban, betrixaban, apixaban, letaxaban (TAK-422), eribaxaban, darexaban, still even more particularly the anticoagulant may be selected from the group consisting of: dabigatran, rivaroxaban, edoxaban, betrixaban, apixaban.

The term “blood sample” as used herein refers to a liquid sample of blood, which comprises or is suspected to comprise an anticoagulant, in particular, a DOAC. Said sample may be an artificial sample, such as reconstituted blood solution comprising or suspected to comprise an anticoagulant, or may be a naturally occurring sample, typically, a blood sample or derivative thereof containing or suspected to contain an anticoagulant, such as blood plasma or a fraction thereof. More typically, the blood sample is a whole blood sample or blood plasma sample or reconstituted blood. The blood sample may comprise anticoagulated and non-anticoagulated samples of the aforementioned. In some aspects, the sample is a blood sample derived from a mammalian species, specifically from a human subject; more specifically from a human subject without any clotting deficiencies such as hemophilia of any kind. Typically, the blood sample comprises physiological amounts of factor II and factor X and of other factors and cofactors relevant for coagulation such as calcium, factor V, etc. See, e.g., O'Donell et al., Br. J. Haematol., 186: 24-36 (2019); Adams and Bird et al., Nephrology, 14: 462-470 (2009); and Palta et al., Indian Journal of Anaesthesia 58(5): 515-523 (2014).

A “physiological amount” as referred to herein is an amount commonly found in a blood sample of a healthy subject, in particular a healthy human subject, more particularly a healthy human adult.

### Step a) of Providing a Composition

In step a) of methods according to some aspects of the present disclosure, a composition is provided comprising: i) thrombin or a prothrombin activator converting prothrombin into thrombin that is factor Xa (FXa) inhibitor insensitive, or mixtures thereof; and ii) FXa or a prothrombin activator converting prothrombin into thrombin that is FXa inhibitor sensitive, or mixtures thereof.

Thrombin, also referred to as factor IIa (FXa) is a well-studied serine protease of the coagulation cascade capable of cleaving peptide substrates at Arg-Gly bonds including natural polypeptides such as fibrinogen as well as synthetic peptides and polypeptides such as Electrozym TH and others, specified in further detail elsewhere herein. Thrombin may be prepared as known to the person skilled in the art; such preparations are commercially available and specified further elsewhere herein.

Thrombin has an enzymatically inactive precursor referred to as prothrombin or factor II. Upon enzymatic cleavage, prothrombin releases thrombin (factor IIa), O'Donell et al., Br. J. Haematol., 186: 24-36 (2019). This enzymatic cleavage is typically performed by a prothrombin activator. The term “prothrombin activator” refers to a substance, in particular an enzyme capable of converting enzymatically inactive prothrombin into thrombin. Typically, the prothrombin activator is a protease, even more typically a serine protease or a metalloprotease. Typical prothrombin activators according to the present disclosure include group A and B snake venom prothrombin activators, such as ecarin, carinactivase, multactivase, in particular ecarin; and Russell's viper venom X (RVV-X) or a related snake venom factor and factor Xa. The conversion of prothrombin into thrombin is typically irreversible and more typically takes place in a fast and quantitative manner, in particular in case the prothrombin activator is supplied in excess. Hence, the enzymatic activities comprised in the composition are, in particular, readily available and lead to predictable thrombin activity that essentially merely depends on the anticoagulant that may be present in the blood sample, in terms of anticoagulant concentration and anticoagulant pharmacological target (inhibiting factor Xa or factor IIa).

In light of the present disclosure, two types of prothrombin activators may be distinguished: i) factor Xa (FXa) inhibitor insensitive prothrombin activators, in particular group A snake venom prothrombin activators, such as ecarin, multactivase; and ii) FXa inhibitor sensitive prothrombin activators. Factor Xa inhibitor sensitive prothrombin activators are considered to act via factor X activation, producing FXa that in turn activates thrombin from its precursor prothrombin. Factor Xa inhibitor sensitive prothrombin activators according to the present disclosure include for example Russell's viper venom X (RVV-X) or a related snake venom factor and FXa. Typically, in the method according to the disclosure, a composition is provided comprising ecarin and FXa.

The composition may typically be present in a dried form. The composition may typically comprise at least one or more of the following: a carrier, a buffer, a substrate capable of detecting thrombin activity, a filling agent, a film forming agent and adhesives and stabilizers. A substrate suitable for detecting thrombin activity according to the present disclosure may be, in particular a peptide substrate such as Electrozym TH (tosyl-glycyl-prolyl-arginyl-p-aminoanilide, Roche Diagnostics GmbH; International Patent Application Pub. No. WO2006/063789 A1), or related tripeptidyl thrombin substrates that are electrochemically detectable including Tos-Gly-Pro-Arg-4-Amino-2-chlorophenol, Bz-IIe-Glu(γ-OH)-Gly-Arg-p-aminoanilide, Bz-IIe-Glu(γ-OCH₃)-Gly-Arg-p-aminoanilide, H-D-Phe-Pip-Arg-p-aminoanilide, Bz-Phe-Val-Arg-p-aminoanilide, H-D-CHG-Ala-Arg-p-aminoanilide. Suitable compositions and ingredients of such dry chemistry reagent compositions are described, e.g., in U.S. Patent Application Pub. Nos. 2003/0146113, 2005/0008537and 2005/0123441.

The composition may be applied in the form of a suspension or in any other solubilized form to a test strip, film or electrode and then dried. In some aspects, this form is advantageous in that it is typically more convenient for use in point-of-care devices.

Further details concerning potential test strips are provided, e.g., in U.S. Pre-grant Pub. No. 2005/0123441 and 2003/0146113, the latter of which describing a particularly suitable electrode system.

### Step b) of Contacting a Blood Sample With the Composition Thereby Generating a Mixture of the Composition With the Blood Sample

In step b) of methods according to some aspects of the present disclosure, a blood sample may be contacted with the composition. The term “contacting” refers to bringing the blood sample in contact with the composition that results in mixing of the composition with the blood sample. Contacting a blood sample with the composition generates a mixture of the composition with the blood sample. “Contacting” may include mixing the composition in liquid form with the liquid blood sample. “Mixing” may be based on mechanical impact such as stirring or shaking or on Brownian motion. However, contacting may also refer to solubilizing the dried composition in the liquid blood sample or vice versa solubilizing the dried blood sample in the liquid composition. Typically, contacting the blood sample, in particular the liquid blood sample, with the composition, solubilizes the thrombin and the prothrombin activator in step b).

Alternatively, both the blood sample and the composition may be present in dried form and the step of contacting a blood sample with the composition may be performed by solubilizing both the blood sample and the composition in a suitable solvent. Typically, in step b), a dried form of the composition is solubilized by the liquid blood sample. More typically, the composition may be immobilized on a suitable carrier, e.g., a carrier which can subsequently be used for or integrated in a detection device such as a test strip, film or an electrode. In some aspects, the composition is present in dried form on a suitable carrier such as a film or test strip and the blood sample may be applied in the vicinity of the composition onto the test strip, film, or onto the electrode.

In some aspects, step b) of contacting the blood sample with the composition generates thrombin activity and/or coagulation in the mixture. This means specifically that the thrombin activity, the FXa activity and the prothrombin activator activities comprised in the composition upon contact with the blood sample lead to an activation of prothrombin present in the blood sample and to an increase in thrombin activity based on the combined amount of prothrombin and thrombin present in the mixture. The resulting measurable thrombin activity in the mixture moreover typically depends on the presence, the type and amount of anticoagulant comprised in the blood sample. The thrombin activity in the mixture may, e.g., be detected over time and be compared to a suitable reference as described elsewhere herein.

The mixture generated by contacting the blood sample with the composition in step b) may comprise at least 0.01 nkat of thrombin activity and at least 0.05 nkat of FXa activity. The thrombin activity may optionally be in the range of 0.01 nkat to 0.04 nkat. The FXa activity may optionally be in the range of 0.05 to 0.2 nkat. In some aspects, the ratio of thrombin activity to FXa activity is in the range of 1:5 to 1:3. In some aspects, the ratio of ecarin activity to FXa activity is in the range of 1:5 to 1:3.

The specified thrombin and FXa activities are advantageous as the combined activities, in particular the specified ratios, allow for a simultaneous determination of all currently available DOACs, as specified elsewhere herein, from a single blood sample in a one-step reaction.

Thrombin activity, factor Xa activity and prothrombin activator activities such as ecarin activity as it may be used in methods of the present disclosure may typically be derived from commercially available enzyme preparations. Suitable thrombin preparations for use in methods according to the present disclosure include, e.g., bovine thrombin, recombinant human thrombin, and thrombin from human plasma. These are commercially available, e.g., from Sigma-Aldrich or Abcam.

Suitable factor Xa preparations for use in methods according to the present disclosure include bovine FXa, human FXa, recombinant human FXa, commercially available for example from Sigma-Aldrich or Abcam. Suitable FXa inhibitor insensitive prothrombin activator preparations for use in the present methods include, e.g., ecarin preparations derived from Echis carinatus venom. One example of a commercially available preparation is from Sigma-Aldrich.

The specified thrombin activities, prothrombin activator activities and/or FXa activities may be adjusted according to the manufacturer's manual for the respective preparations. Moreover, there are tests available allowing the determination of these activities as specified elsewhere herein.

The term “thrombin activity” as referred to herein typically relates to an enzymatic activity that is capable of catalyzing the release of fibrin from its precursor fibrinogen. Thrombin is a well-studied serine protease capable of cleaving peptide substrates at Arg-Gly bonds including natural polypeptides such as fibrinogen as well as synthetic peptides and polypeptides such as Electrozym TH (Roche Diagnostics GmbH) and others, specified in further detail elsewhere herein. In particular, synthetic peptides carrying the amino acid motif “Gly-Pro-Arg” may be cleaved. As used herein, the term “thrombin activity” refers to the enzymatic activity of thrombin capable of cleaving fibrinogen and related peptides and polypeptides, natural and synthetic, at Arg-Gly bonds; in particular the term refers to the serine proteolytic activity in class EC 3.4.21. As used herein, “thrombin activity” includes the activity of prothrombin activators converting prothrombin into thrombin that are FXa inhibitor insensitive including group A and group B snake venom prothrombin activators, such as ecarin, carinacitvase, multactivase, and ecarin.

Thrombin activity, FXa activity and prothrombin activator activity, such as ecarin activity, may in principle be determined using any known method in the art capable of detecting proteolytic activity. Typically, these tests include antibody-based detection like enzyme-linked immunosorbent assay (ELISA) tests, fluorometric or colorimetric assays based on fluorophore or chromophore release upon thrombin mediated cleavage of a synthetic substrate and the like. Thrombin activity tests are commercially available, including ELISA kits and other chromogenic tests from Abcam or Simga-Aldrich. Ecarin or Ecarin Clotting Time (ECT) tests are commercially available, e.g. from Stago. Factor Xa (FXa) activity tests are also commercially available from Abcam or Sigma-Aldrich. Determining thrombin activity in a method according to the disclosure typically involves using a substrate that is cleaved by the proteolytic activity of thrombin. Further details are specified elsewhere herein.

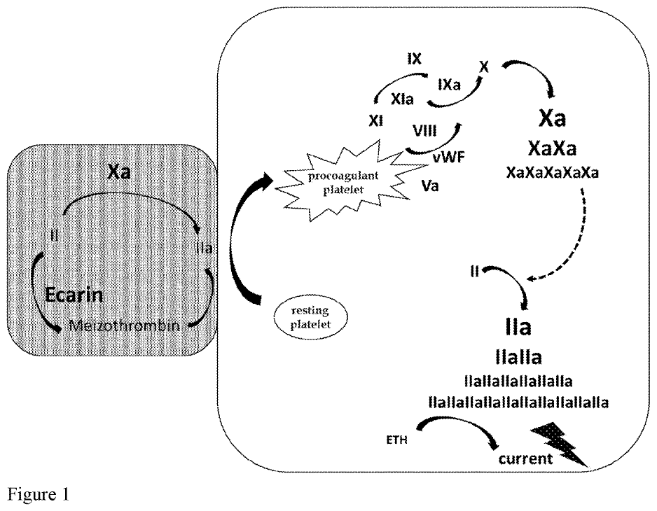

In healthy mammalian tissues, thrombin activity is usually performed by endogenous thrombin (factor IIa). Typically, said enzymatic reaction can be inhibited by an anticoagulant referred to as an inhibitor of the factor IIa (thrombin) inhibitor type. An overview of the clotting cascade and the detection principle implemented by certain methods and other inventive aspects of the present disclosure is depicted in FIG. 1.

Factor Xa is a prothrombin activating protease capable of releasing thrombin from prothrombin. The term “factor Xa activity” refers to an enzymatic activity that catalyzes said release of thrombin (factor IIa) from its precursor prothrombin (factor II). Typically, said enzymatic reaction may be inhibited by an anticoagulant referred to as an inhibitor of the factor Xa inhibitor type.

The term “inhibitor” as used herein is to be given its ordinary and customary meaning to a person of ordinary skill in the art and is not to be limited to a special or customized meaning. The term specifically may refer, without limitation, to at least a substance or material that is capable of inhibiting a given chemical reaction, in particular an enzymatic reaction including proteolytic cleavage of clotting factors, more particularly proteolytic cleavage of prothrombin and factor X. Even more particularly, said inhibitor is an anticoagulant of the factor IIa inhibitor type and/or an anticoagulant of the factor Xa inhibitor type.

### Step c) of Measuring Thrombin Activity Using a Substrate Capable of Detecting Thrombin Activity

In step c) of methods according to some aspects of the present disclosure, thrombin activity may be measured by using a substrate capable of detecting thrombin activity as specified in further detail elsewhere herein. In some aspects, the substrate is a synthetic peptide with a label that allows for electrochemically detection, in particular amperometric detection, such as Electrozym TH (Roche Diagnostics GmbH) and related tripeptidyl thrombin substrates that are electrochemically detectable including Tos-Gly-Pro-Arg-4-Amino-2-chlorophenol, Bz-IIe-Glu(γ-OH)-Gly-Arg-p-aminoanilide, Bz-IIe-Glu(γ-OCH₃)-Gly-Arg-p-aminoanilide, H-D-Phe-Pip-Arg-p-aminoanilide, Bz-Phe-Val-Arg-p-aminoanilide, and/or H-D-CHG-Ala-Arg-p-aminoanilide. Alternatively, labels that are optically detectable may be used. Suitable optically detectable substrates include, e.g., the above-mentioned peptides with a p-nitroanilide moiety that after proteolytical cleavage generate nitroaniline. The synthetic peptide may, in some aspects, be cleaved into a residual peptide and a detectable label, e.g., an electrochemically detectable label or an optically detectable label. A suitable electrochemically detectable label is phenylenediamine, which may for example be proteolytically released from Electrozyme TH. Release of the electrochemically detectable label typically generates an electrical signal which may be measured by electrochemical detection, optionally, such measurement may be performed over time. The kinetics of thrombin generation and accumulation in the blood sample is dependent on the DOAC concentration and anticoagulant pharmacological target (inhibiting factor Xa or factor IIa). Release of an optically detectable label typically generates an optical signal which may be detected by optical means.

In some aspects, methods according to the present disclosure utilize electrochemical detection, e.g., amperometric detection, of thrombin activity over time. Thrombin activity measurements based on electrochemically detectable labels may be determined by electrode arrangements such as thin layer electrodes suitable for voltametric and, optionally, amperometric measurements, as described e.g., in U.S. Pre-grant Pub. Nos. 2003/0146113 or 2005/0123441, or as implemented in the commercially available CoaguChek systems of Roche Diagnostics GmbH. The cleavage of an amperogenic substrate by thrombin activity may lead to an electric current flow. Said current may typically be related to the amount of thrombin in the blood sample.

In some aspects, thrombin activity may be measured at regular intervals such as every 10 or every 20 seconds after generating the mixture of the composition with the blood sample, or may be measured continuously. More particularly, the thrombin activity may be measured at regular intervals starting from 30 seconds after generating the mixture. In the alternative, the thrombin activity may be measured at fixed points in time after generating said mixture, such as after 60 s, 100 s, and 150 s. Other fixed time points may be measured. Typically, the measurements are performed to determine the point in time when a certain threshold of thrombin activity has been reached (clotting time, CT). In the case of amperometric thrombin activity measurements, said threshold may specifically refer to a current value, such as 50 nA, generated by thrombin activity from an electrochemically detectable, in particular an amperogenic, substrate.

### Step d) Comparing the Measured Thrombin Activity to a Reference

In step d) of methods according to some aspects of the present disclosure, the measured thrombin activity may be compared to a reference. Typically, the reference is a predetermined reference such as predetermined measurement data from a blood sample known to be free of any anticoagulants, e.g., DOACs. In some aspects, comparing the measured thrombin activity to a reference refers to comparing the time at which a certain threshold in thrombin activity has been reached in the sample with a reference such as a reference time. In some aspects, comparing the measured thrombin activity to a reference refers to comparing at a given time point the thrombin activity measured in the sample with a reference, e.g. with a reference thrombin activity value or a threshold value.

The term “comparing” as used herein encompasses comparing the thrombin activity measured in the sample with the thrombin activity measured in a reference specified elsewhere in this description.

It is to be understood that comparing as used herein refers to a comparison of corresponding parameters or values, e.g., an absolute amount is compared to an absolute reference amount while an activity is compared to a reference activity and activity over time is compared to activity over time.

The comparison referred to in step d) of selected methods of the present disclosure may be carried out manually or computer assisted. For a computer assisted comparison, the value of the measured thrombin activity may be compared to values corresponding to suitable references which are stored in a database by a computer program. The computer program may further evaluate the result of the comparison, i.e. automatically provide the desired assessment in a suitable output format. Based on the comparison of the thrombin activity measured in step c) and the reference, it is possible to determine the anticoagulant in the sample.

The term “reference” as used herein refers to a thrombin activity value which allows for allocation of a sample into either the group of blood samples that are free of anticoagulants, in particular free of a significant amount of anticoagulants, or that comprise a significant amount of anticoagulants. Hence, a “reference” may refer to a specified amount of thrombin or to a specified thrombin activity detectable in a blood sample that is free of anticoagulants, also referred to herein as a naïve sample. Typically, a “reference” may refer to a thrombin activity that can be related to an amount of thrombin present in said naïve sample. Said thrombin activity may refer to an electrochemically determined activity, in particular to an activity determined by amperometric measurements.

Such a reference thrombin activity value can be a threshold which separates these groups from each other. In particular, the reference value may be a value that allows additionally for distinguishing between different types of anticoagulants, particularly between direct oral anticoagulants of the factor IIa (thrombin) inhibitor type or of the FXa inhibitor type, in particular when the thrombin activity is measured at regular intervals or continuously.

Typically, said reference is derived from a blood sample of a healthy subject or a group of healthy subjects known to not have taken any anticoagulants including DOACs. More typically said subject or group of subjects are human beings, in particular human beings known not to suffer from any clotting deficiency such as hemophilia. The reference amount applicable for an individual subject may vary depending on various physiological parameters such as age, gender, or subpopulation.

A “significant amount of anticoagulant” typically refers to an amount of at least 30 ng/mL or of at least 50 ng/mL of a DOAC in a blood sample. The significant amount of anticoagulant is typically the amount of DOAC in a blood sample of a patient above which surgical intervention is no longer considered safe. “Free of anticoagulant” typically refers to an amount of a DOAC below 30 ng/mL (Shaw et al. 2020, Med Clin North Am. 104(4):709-726).

Hence, methods according to the present disclosure are advantageous as they enable simultaneous determination of the presence of a significant amount of all currently available DOACs from a single blood sample in a one-step reaction.

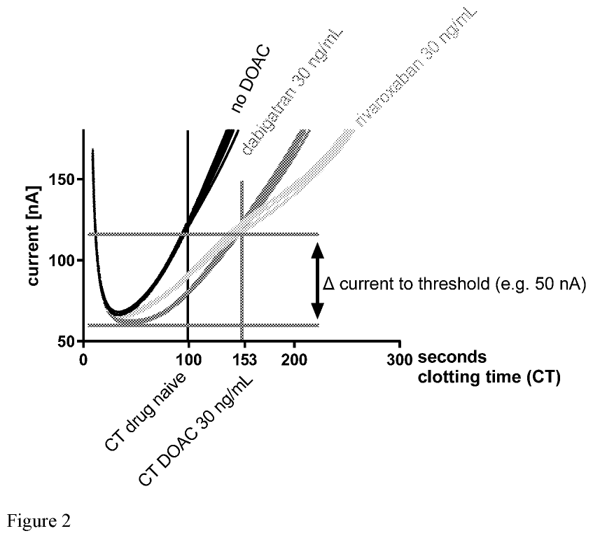

Typically, in step d) thrombin activity measured over time is compared to the thrombin activity of a reference measured over time. More typically, the reference is a calibration data set with thrombin activity over time with known concentrations of factor Xa and factor IIa inhibitors. Still more typically, step d) comprises determining the slope in the measured current over time at selected time points to that of a reference, in particular in a multivariate analysis (see for example FIG. 2).

### Step e) Determining the Anticoagulant Based on the Comparison

In step e) of methods according to some aspects of the present disclosure, the anticoagulant is determined based on the prior comparison. In some aspects, determining the presence of the at least one anticoagulant comprises determining the amount of the at least one anticoagulant. The determining may, e.g., involve comparing the amperometric measurements over time to reference measurements over time.

Step e) may further comprise discriminating between factor IIa inhibitor type and factor Xa inhibitor type anticoagulants. Still more particularly, discriminating between factor IIa and factor Xa inhibitor type anticoagulants may comprise comparing the amperometric measurements over time to reference measurements over time, optionally by multivariate data analysis.

In some aspects, the amperometric measurements and signal generation depend on the accumulated thrombin in the sample and thus on the amount of anticoagulant and may also reflect the mode of inhibition (factor Xa or factor IIa inhibition) that may slow down thrombin accumulation in the test device.

In some aspects, determining the anticoagulant based on the comparison comprises assessing whether the anticoagulant is present in the sample within a certain threshold, in particular up to 50 ng/ml of the sample, or not. Hence, in line with the present disclosure the threshold may relate to a certain amount of anticoagulant; more particularly, the threshold relates to a certain thrombin activity detectable as a current generated from an amperogenic substrate in a sample that is free of significant amounts of anticoagulant, e.g. in a naïve sample.

Typically, when applying a threshold of generated current (corresponding to a defined amount of thrombin generated) the time until the threshold is reached is dependent on the anticoagulant, e.g. DOAC, concentration. In particular, the higher the anticoagulant, e.g. DOAC, level, the more thrombin is inhibited and the longer it takes to generate the threshold current (e.g. 50 nA). Hence, specifically in case the current measurements in the sample after a certain time are below the threshold, this indicates the presence of an anticoagulant in a significant amount. More specifically, in case the current measurements in the sample after a certain time meet the threshold or are above the threshold, this indicates the absence of an anticoagulant or presence of an anticoagulant in a non-significant amount.

In some aspects, additional currents measured at selected time points may provide additional information if a factor IIa inhibitor or a factor Xa inhibitor is present. This may be based on the fact that factor Xa is downstream of factor IIa in the blood coagulation cascade model and the curves between the drug classes typically are kinetically different. An example is given in FIG. 2. In this figure, the type of anticoagulant (factor IIa inhibitor or factor Xa inhibitor) may be identified e.g. with an additional measurement at 200 seconds. At this additional time point, the current measured in a sample comprising dabigatran (a factor IIa inhibitor) is higher than the current measured in a sample comprising rivaroxaban (a factor Xa inhibitor), indicating the different modes of actions of these anticoagulants within the coagulation cascade and thereby their distinguishable effects on the time-dependent generation of the current signal caused by the thrombin activity.

The present disclosure further contemplates a working electrode of an analyte sensor, wherein the surface of the working electrode comprises at least a first reaction zone providing a composition comprising: i) thrombin or a prothrombin activator converting prothrombin into thrombin that is factor Xa (FXa) inhibitor insensitive, or mixtures thereof; and ii) FXa or a prothrombin activator converting prothrombin into thrombin that is FXa inhibitor sensitive, or mixtures thereof; wherein said first reaction zone is configured for contacting the composition with a blood sample suspected to comprise an anticoagulant for a time and under conditions which allow for thrombin activation and/or factor X activation in the blood sample; further comprising at least a second reaction zone comprising a substrate capable of detecting thrombin activity.

In some aspects, the first reaction zone comprises the composition in a dried form. “Dried form” as specified herein may relate to all kinds of dry forms of a composition including for example immobilization of the composition on a solid substrate or a solid carrier, freeze-dried state of the composition, dehydrated solid form of the composition and the like; specifically “dried form” of the composition may relate to the composition being immobilized on a solid substrate or a solid carrier. A solid substrate or a solid carrier according to the present disclosure includes, e.g., a test strip, a film, or an electrode.

The second reaction zone may comprise an electrochemically detectable substrate capable of detecting thrombin activity. Electrochemically detectable substrates may be determined by electrode arrangements such as thin layer electrodes suitable for voltametric and, typically, amperometric measurements.

Moreover, the present disclosure relates to a device for determining an anticoagulant in a blood sample comprising: a) an analyzing unit comprising (i) a first reaction zone providing a composition comprising i) thrombin or a prothrombin activator converting prothrombin into thrombin that is FXa inhibitor insensitive, or mixtures thereof; and ii) FXa or a prothrombin activator converting prothrombin into thrombin that is FXa inhibitor sensitive, or mixtures thereof; wherein said first reaction zone is configured for contacting the composition with a blood sample suspected to comprise an anticoagulant for a time and under conditions which allow for thrombin activation in the blood sample; (ii) a second reaction zone comprising a substrate capable of detecting thrombin activity, in particular an electrochemically detectable substrate; and (iii) a detector which is capable of determining substrate turnover in the blood sample, thereby measuring thrombin activity; and b) an evaluation unit which is capable of determining the anticoagulant based on the measured thrombin activity by comparison to a reference.

The term “device” as used herein relates to a system comprising the aforementioned units operatively linked to each other as to allow the determination of one or more anticoagulants according to methods of the present disclosure.

The analyzing unit may comprise a first reaction zone providing the composition in immobilized form on a solid support or carrier, which is to be contacted with the blood sample comprising anticoagulant or suspected to comprise anticoagulant. More typically, in the first reaction zone, conditions apply that allow of the generation of a mixture of the composition with the blood sample. Moreover, in the first reaction zone, it is possible to apply conditions, which allow for thrombin activation and/or factor X activation in the blood sample or in the mixture of the composition and the blood sample.

Furthermore, the analyzing unit may comprise a second reaction zone comprising an electrochemically detectable substrate capable of detecting thrombin activity. Electrochemically detectable substrates may be determined by electrode arrangements such as thin layer electrodes suitable for voltametric and, typically, amperometric measurements.

The analyzing unit may further comprise a detector capable of determining substrate turnover in a blood sample.

The detector may be adapted to detect determine the amount of substrate turnover, e.g., by detecting the electrical signal generated by thrombin activity on the electrochemically detectable substrate. The determined amount may be subsequently transmitted to the evaluation unit. Said evaluation unit may comprise a data processing element, such as a computer, with an implemented algorithm for determining the anticoagulant present in the blood sample based on the measured thrombin by comparison to a reference specified further elsewhere herein.

The present disclosure also relates to an analyte sensor for detecting an anticoagulant in a blood sample by electrochemical detection, comprising at least one working electrode as specified elsewhere herein, and at least an evaluation unit, which is capable of determining the anticoagulant based on the measured thrombin activity by comparison to a reference.

The analyte sensor may comprise, e.g., at least two electrodes of which at least one electrode is a so-called working electrode. The electrodes may be composed of any conventional electrode materials such as metals, noble metals, alloys or graphite and are preferably composed of noble metals such as gold or palladium, or graphite. The various electrodes of the sensor can be composed of the same or different materials. In some aspects, the electrodes are made of palladium.

The present disclosure further relates to a kit for determining an anticoagulant in a blood sample comprising a composition and a substrate capable of detecting thrombin activity, wherein the composition comprises: i) thrombin or a prothrombin activator converting prothrombin into thrombin that is FXa inhibitor insensitive, or mixtures thereof; and ii) FXa or a prothrombin activator converting prothrombin into thrombin that is FXa inhibitor sensitive, or mixtures thereof; and wherein the substrate is an electrochemically detectable substrate capable of detecting thrombin activity, in particular an electrochemically detectable peptide substrate such as Electrozym TH.

The term “kit,” as used herein, refers to a collection of the aforementioned components, typically, provided in separately or within a single container. The container also typically comprises instructions for carrying out methods of the present disclosure. These instructions may be in the form of a manual or may be provided by a computer program code which is capable of carrying out or supports the determination of an anticoagulant referred to in the methods of the present disclosure when implemented on a computer or a data processing device. The computer program code may be provided on a data storage medium or device such as an optical storage medium (e.g., a Compact Disc) or directly on a computer or data processing device or may be provided in a download format such as a link to an accessible server or cloud. Moreover, the kit may, usually, comprise standards for reference amounts of thrombin and prothrombin activities for calibration purposes as described elsewhere herein in detail. Typically, the reference is a calibration data set with thrombin activity over time with known concentrations of factor Xa and factor IIa inhibitors. The kit according to the present disclosure may also comprise further components which are necessary for carrying out methods of the disclosure such as solvents, washing solutions and/or reagents required for detection of the anticoagulant. Further, it may comprise a device according to the disclosure, either in parts or in its entirety.

The aforementioned kit of the present disclosure may be for use in assessing an anticoagulant in a blood sample of a subject.

Moreover, the present disclosure contemplates the use of a composition providing at least 0.01 nkat of thrombin activity and at least 0.05 nkat of factor Xa (FXa) activity for determining an anticoagulant in a blood sample.

It is to be understood that the definitions and explanations of the terms above apply for all embodiments described in this specification and the accompanying claims. The following embodiments are non-limiting embodiments envisaged according to the present disclosure:

Embodiment 1. A method for determining an anticoagulant in a blood sample, the method comprising the following steps:
a) providing a composition comprising:
   i) thrombin or a prothrombin activator converting prothrombin into thrombin that is factor Xa (FXa) inhibitor insensitive, or mixtures thereof; and
ii) FXa or a prothrombin activator converting prothrombin into thrombin that is FXa inhibitor sensitive, or mixtures thereof;
b) contacting a blood sample with the composition thereby generating a mixture of the composition with the blood sample, wherein the mixture comprises at least 0.01 nkat of thrombin activity and at least 0.05 nkat of FXa activity;
c) measuring thrombin activity using a substrate capable of detecting thrombin activity;
d) comparing the measured thrombin activity to a reference; and
e) determining the anticoagulant based on the comparison.

Embodiment 2. The method according to the preceding embodiment, wherein the anticoagulant is an oral anticoagulant; in particular a direct oral anticoagulant (DOAC).

Embodiment 3. The method according to any one of the preceding embodiments, wherein the anticoagulant is a direct oral anticoagulant of the factor IIa (thrombin) inhibitor type or of the FXa inhibitor type.

Embodiment 4. The method according to any one of the preceding embodiments, wherein the anticoagulant is a direct oral anticoagulant selected from the group consisting of: dabigatran, rivaroxaban, edoxaban, betrixaban, apixaban, TAK-422, eribaxaban, darexaban, particularly selected from the group consisting of: dabigatran, rivaroxaban, edoxaban, betrixaban, apixaban.

Embodiment 5. The method according to any one of the preceding embodiments, wherein the composition comprises: I) ecarin; and II) FXa.

Embodiment 6. The method of the preceding embodiment, wherein the composition comprises an ecarin activity in the range of 0.01-0.04 nkat and/or a FXa activity in the range of 0.05-0.2 nkat; optionally, wherein the ratio of ecarin activity to FXa activity is in the range of 1:5 to 1:3.

Embodiment 7. The method according to any one of the preceding embodiments, wherein the prothrombin activator is provided in dried form; and/or wherein contacting the blood sample with the composition solubilizes the prothrombin activator in step b).

Embodiment 8. The method according to any one of the preceding embodiments, wherein the blood sample is a sample selected from the group consisting of: whole blood, plasma, and reconstituted blood; in particular the blood sample comprises anticoagulated and non-anticoagulated samples of the aforementioned.

Embodiment 9. The method according to any one of the preceding embodiments, wherein the blood sample is a blood sample derived from a mammalian species, in particular from a human; more particularly from a human without any clotting deficiencies such as hemophilia.

Embodiment 10. The method according to any one of the preceding embodiments, wherein the FXa inhibitor insensitive prothrombin activator comprises a prothrombin activator of group A snake venom prothrombin activators, such as ecarin, carinacitvase, multactivase, in particular ecarin; and/or wherein the FXa inhibitor sensitive prothrombin activator comprises Russell's viper venom X (RVV-X) or a related snake venom factor or factor Xa.

Embodiment 11. The method according to any one of the preceding embodiments, wherein the reference is a predetermined reference such as predetermined measurement data from a sample known to be free of any anticoagulants, in particular free of DOACs.

Embodiment 12. The method according to any one of the preceding embodiments, wherein step b) contacting the blood sample with the composition generates thrombin activity and/or coagulation in the mixture.

Embodiment 13. The method according to any one of the preceding embodiments, wherein step c) of measuring thrombin activity comprises electrochemical detection of thrombin activity by a suitable substrate, in particular a peptide substrate such as Electrozym TH (tosyl-glycyl-prolyl-arginyl-p-aminoanilide), or related tripeptidyl thrombin substrates that are electrochemically detectable Tos-Gly-Pro-Arg-4-Amino-2-chlorophenol, Bz-IIe-Glu(γ-OH)-Gly-Arg-p-aminoanilide, Bz-IIe-Glu(γ-OCH₃)-Gly-Arg-p-aminoanilide, H-D-Phe-Pip-Arg-p-aminoanilide, Bz-Phe-Val-Arg-p-aminoanilide,, H-D-CHG-Ala-Arg-p-aminoanilide.

Embodiment 14. The method according to any one of the preceding embodiments, wherein step c) of measuring thrombin activity comprises amperometric detection over time.

Embodiment 15. The method according to any one of the preceding embodiments, wherein step d) of comparing the measured thrombin activity, in particular the measured thrombin activity over time, to the thrombin activity of a reference, in particular to the thrombin activity over time of a reference; in particular wherein step d) comprises determining the slope in the measured current over time to that of a reference.

Embodiment 16. The method according to any one of the preceding embodiments, wherein step e) comprises determining the presence of the at least one anticoagulant, the amount of the at least one anticoagulant, and/or discriminating between factor IIa inhibitor type and factor Xa inhibitor type anticoagulants.

Embodiment 17. The method according to the preceding embodiment, wherein discriminating between factor IIa and factor Xa inhibitor type anticoagulants comprises comparing the amperometric measurings over time to reference measurings over time, in particular by multivariate data analysis.

Embodiment 18. The method according to any one of the preceding embodiments, wherein step e) of determining the anticoagulant based on the comparison comprises assessing whether the anticoagulant is present in the sample within a certain threshold, in particular up to 50 ng/ml of the sample, or not.

Embodiment 19. A working electrode of an analyte sensor, wherein the surface of the working electrode comprises at least a first reaction zone providing a composition comprising: i) thrombin or a prothrombin activator converting prothrombin into thrombin that is factor Xa (FXa) inhibitor insensitive, or mixtures thereof; and ii) FXa or a prothrombin activator converting prothrombin into thrombin that is FXa inhibitor sensitive, or mixtures thereof; wherein said first reaction zone is configured for contacting the composition with a blood sample suspected to comprise an anticoagulant for a time and under conditions which allow for thrombin activation and/or factor X activation in the blood sample; further comprising at least a second reaction zone comprising a substrate capable of detecting thrombin activity.

Embodiment 20. The working electrode of Embodiment 19, wherein the first reaction zone comprises the composition in a dried form.

Embodiment 21. A device for determining an anticoagulant in a blood sample comprising:
a) an analyzing unit comprising
   (i) a first reaction zone providing a composition comprising
      i) thrombin or a prothrombin activator converting prothrombin into thrombin that is FXa inhibitor insensitive, or mixtures thereof; and
      ii) FXa or a prothrombin activator converting prothrombin into thrombin that is FXa inhibitor sensitive, or mixtures thereof; wherein said first reaction zone is configured for contacting the composition with a blood sample suspected to comprise an anticoagulant for a time and under conditions which allow for thrombin activation in the blood sample;
   (ii) a second reaction zone comprising a substrate capable of detecting thrombin activity, in particular an electrochemically detectable substrate; and
   (iii) a detector which is capable of determining substrate turnover in the blood sample, thereby measuring thrombin activity;
b) an evaluation unit which is capable of determining the anticoagulant based on the measured thrombin activity by comparison to a reference.

Embodiment 22. An analyte sensor for detecting an anticoagulant in a blood sample by electrochemical detection, comprising at least one working electrode as in Embodiment 21, and at least an evaluation unit, which is capable of determining the anticoagulant based on the measured thrombin activity by comparison to a reference.

Embodiment 23. A kit for determining an anticoagulant in a blood sample comprising a composition and a substrate capable of detecting thrombin activity, wherein the composition comprises: i) thrombin or a prothrombin activator converting prothrombin into thrombin that is FXa inhibitor insensitive, or mixtures thereof; and ii) FXa or a prothrombin activator converting prothrombin into thrombin that is FXa inhibitor sensitive, or mixtures thereof; and wherein the substrate is an electrochemically detectable substrate capable of detecting thrombin activity, in particular an electrochemically detectable peptide substrate such as Electrozym TH.

Embodiment 24. Use of a composition providing at least 0.01 nkat of thrombin activity and at least 0.05 nkat of factor Xa (FXa) activity for determining an anticoagulant in a blood sample.

Embodiment 25. A method for determining an anticoagulant in a blood sample, comprising a step of: contacting a blood sample with a composition, thereby generating a mixture of the composition with the blood sample; wherein the composition comprises i) thrombin or a prothrombin activator converting prothrombin into thrombin that is factor Xa (FXa) inhibitor insensitive, or mixtures thereof; and/or ii) FXa or a prothrombin activator converting prothrombin into thrombin that is FXa inhibitor sensitive, or mixtures thereof; and wherein the mixture comprises at least 0.01 nkat of thrombin activity and at least 0.05 nkat of FXa activity.

The content of any patent, patent application, and/or publication cited in this specification is incorporated by reference in its entirety, with the exception of lexicographical definitions used therein. Any lexicographical definition in a cited patent, patent application, or publication that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

### EXAMPLES

The following Examples illustrate aspects of the present disclosure. These Examples shall not be construed whatsoever as limiting its scope.

### Example 1: DOAC Determination Using Amperometric Detection

An assay for determining the presence of DOACs blood samples was conducted using FXa and ecarin as activators. Ecarin activity was adjusted to 0.025 nkat and FXa activity was adjusted to 0.1 nkat. The samples were adjusted to contain 30 ng/mL of either dabigatran (FIIa inhibitor type) or rivaroxaban (FXa inhibitor type) in whole blood. The reference was whole blood without DOAC addition. Amperometric measurements were conducted using a CoaguChek point-of-care coagulometer.

The results are depicted in FIG. 2. In untreated blood in absence of a DOAC, the current reaches a minimum when the blood sample dissolves the assay chemistry. Then the activators in the assay chemistry generated thrombin, which cleaves the amperogenic thrombin substrates and generates the current. In un-inhibited state, the current is generated rapidly. In presence of an inhibitor, the current generation is slower. The time after a certain current threshold is generated (e.g. 50 nA) after the minimum is the clotting time. The clotting time is one of the measurands that is dependent on the amount of anticoagulation, the dose of DOAC in the blood sample. In this example, a current threshold of 50 nA of the DOAC-free has been already received after about 100 seconds, whereby the same current threshold of 50 nA is received only after about 153 seconds both for samples containing 30 ng/mL of either dabigatran (FIIa inhibitor type) or rivaroxaban (FXa inhibitor type). At the time point of 100 seconds, both for samples containing 30 ng/mL of either dabigatran or rivaroxaban the respective current values of these DOAC samples are between 20-30 nA. Therefore, in this example, a current signal measurement after about 100 seconds can be used to discriminate between a DOAC-free sample (having a current signal of about 50 nA after 100 seconds) and DOAC-containing samples (having much lower currents signals of about 20-30 nA after 100 seconds).

The shape, i.e. time-dependent signal changes, of the curves allows in this example also to additionally discriminate between factor IIa inhibitor type and factor Xa inhibitor type anticoagulants. While anticoagulation by dabigatran leads to an initial delay in the clotting time and subsequent step rise comparable to the rise in the curve of the reference, coagulation in the presence of rivaroxaban is generally slowed down as can be seen by the decrease in the steepness of the curve and appears to multiphasic in contrast to dabigatran monophasic inhibition.

### Example 2: Assessing a Dose-Response Curve for Presently Available DOACS

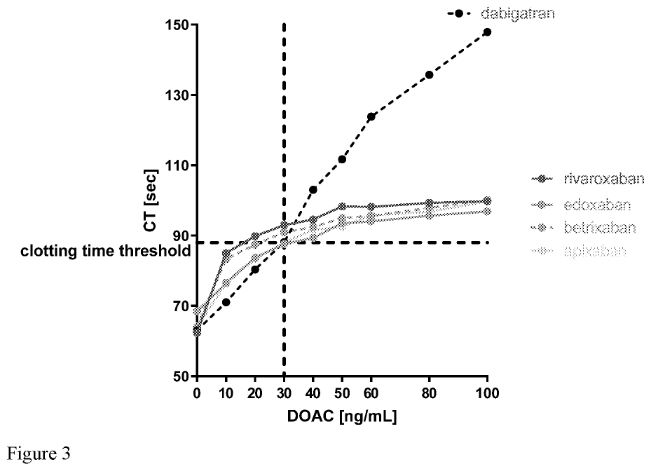

The assay set up was essentially as in Example 1. The indicated concentrations of DOACs (on the x-axis) were added to anticoagulant-naïve whole blood and the assay contained FXa and ecarin with ecarin activity adjusted to 0.025 nkat and FXa activity adjusted to 0.1 nkat. The results are illustrated by FIG. 3.

The clotting time to reach the predefined current threshold (as described in FIG. 2 is on the y-axis. The higher the concentration of an anticoagulant, the longer is the clotting time, the longer it takes to reach the current threshold, because thrombin is inhibited. The dose-reaction curve for the factor Xa inhibitors is very similar to each other, however, the factor IIa inhibitor dabigatran has a distinct dose dependency. By adjusting the balance of the activators (e.g. factor Xa inhibitor insensitive thrombin activator ecarin and factor Xa sensitive thrombin activator FXa), the relative sensitives of the dabigatran dose response curve and the FXa inhibitor dose response curves can be shifted relative to each other. This means that a particularly useful configuration is possible, in which dabigatran at 30 ng/mL and FXa inhibitors at 30 ng/mL will have identical ranges of clotting times. This forms the basis for a generic DOAC test that is negative if the DOAC is below 30 ng/mL (uncalibrated clotting times less than 89 seconds) and positive for blood samples with DOAC levels above 30 ng/mL (uncalibrated clotting times more than 89 seconds). By adjusting the balance of the activators (e.g. factor Xa inhibitor insensitive thrombin activator ecarin and factor Xa inhibitor sensitive thrombin activator FXa), the overlap point can be set to correspond to 50 ng/mL DOAC if desired.

### CITED LITERATURE

1. Adams R.L.C. and Bird, R.J. (2009), Review Article: “Coagulation Cascade and Therapeutics Update: Relevance to Nephrology. Part 1: Overview of Coagulation, Thrombophilias and History of Anticoagulants.” Nephrology, 14: 462-470. https://doi.org/10.1111/j.1440-1797.2009.01128.x.
2. Harris L.F., Castro-López V., Killard A. J., 2013, “The Development of the Heparin Monitoring System Based on Microfluidics Technology.” Trends in Analytical Chemistry 50: 85-95. http://dx.doi.org/10.1016/j. trac.2013.05.009.
3. O'Donnell, J.S., O'Sullivan, J.M. and Preston, R.J.S. (2019), “Advances in Understanding the Molecular Mechanisms That Maintain Normal Haemostasis.” Br J Haematol, 186: 24-36. https://doi. org/10.1111/bjh.15872.
4. Palta, S., Saroa, R., & Palta, A. (2014). “Overview of the Coagulation System.” Indian J. Anaesthesia, 58(5), 515-523. https://doi.org/10.4103/0019-5049.144643.
5. Shaw JR, Kaplovitch E, Douketis J., “Periprocedural Management of Oral Anticoagulation.” Med Clin North Am. 2020 Jul;104(4):709-726. doi: 10.1016/j.mcna.2020.02.005).

1. A method for determining an anticoagulant in a blood sample, the method comprising the following steps:
a) providing a composition comprising:
i) thrombin or a prothrombin activator converting prothrombin into thrombin that is factor Xa (FXa) inhibitor insensitive, or mixtures thereof; and
ii) FXa or a prothrombin activator converting prothrombin into thrombin that is FXa inhibitor sensitive, or mixtures thereof;
b) contacting a blood sample with the composition thereby generating a mixture of the composition with the blood sample, wherein the mixture comprises at least 0.01 nkat of thrombin activity and at least 0.05 nkat of FXa activity;
c) measuring thrombin activity using a substrate capable of detecting thrombin activity;
d) comparing the measured thrombin activity to a reference; and
e) determining the anticoagulant based on the comparison.

2. The method of claim 1, wherein the anticoagulant is an oral anticoagulant; optionally, a direct oral anticoagulant (DOAC); optionally a direct oral anticoagulant of the factor IIa (thrombin) inhibitor type or of the FXa inhibitor type.

3. The method of claim 2, wherein the anticoagulant is selected from the group consisting of: dabigatran, rivaroxaban, edoxaban, betrixaban, apixaban, TAK-422, eribaxaban, and darexaban.

4. The method of claim 1, wherein the FXa inhibitor insensitive prothrombin activator comprises a prothrombin activator of group A snake venom prothrombin activators, optionally ecarin, carinacitvase, multactivase; and/or wherein the FXa inhibitor sensitive prothrombin activator comprises Russell's viper venom X (RVV-X) or a related snake venom factor or FXa.

5. The method of claim 1, wherein the composition comprises: i) ecarin; and ii) FXa.

6. The method of claim 5, wherein the composition comprises an ecarin activity in the range of 0.01-0.04 nkat and/or a FXa activity in the range of 0.05-0.2 nkat; typically wherein the ratio of ecarin activity to FXa activity is in the range of 1:5 to 1:3.

7. The method of claim 1, wherein the reference is a predetermined reference, optionally predetermined measurement data from a sample known to be free of i) any anticoagulants, and/or ii) DOACs.

8. The method of claim 1, wherein step b) contacting the blood sample with the composition generates thrombin activity and/or coagulation in the mixture.

9. The method of claim 1, wherein step c) of measuring thrombin activity comprises electrochemical detection of thrombin activity using one or more suitable substrates, optionally a peptide substrate comprising Electrozym TH (tosyl-glycyl-prolyl-arginyl-p-aminoanilide), or related tripeptidyl thrombin substrates that are electrochemically detectable, Tos-Gly-Pro-Arg-4-Amino-2-chlorophenol, Bz-IIe-Glu(γ-OH)-Gly-Arg-p-aminoanilide, Bz-IIe-Glu(γ-OCH₃)-Gly-Arg-p-aminoanilide, H-D-Phe-Pip-Arg-p-aminoanilide, Bz-Phe-Val-Arg-p-aminoanilide, and/or H-D-CHG-Ala-Arg-p-aminoanilide.

10. The method of claim 1, wherein step c) of measuring thrombin activity comprises amperometric detection over time.

11. The method of claim 1, wherein step d) comprises:
i) comparing the measured thrombin activity to the thrombin activity of a reference; and/or
ii) comparing the measured thrombin activity over time, to the thrombin activity over time of a reference.

12. The method of claim 1, wherein step e) comprises determining the presence of the anticoagulant and/or the amount of the at least one anticoagulant.

13. The method of claim 1, wherein step e) of determining the anticoagulant based on the comparison comprises assessing whether the anticoagulant is present in the sample within a certain threshold, optionally up to 50 ng/ml of the sample, or not.

14. A working electrode of an analyte sensor, wherein the surface of the working electrode comprises at least a first reaction zone providing a composition comprising:
i) thrombin or a prothrombin activator converting prothrombin into thrombin that is factor Xa (FXa) inhibitor insensitive, or mixtures thereof; and
ii) FXa or a prothrombin activator converting prothrombin into thrombin that is FXa inhibitor sensitive, or mixtures thereof;
wherein said first reaction zone is configured for contacting the composition with a blood sample suspected to comprise an anticoagulant for a time and under conditions which allow for thrombin activation and/or factor X activation in the blood sample; further comprising at least a second reaction zone comprising a substrate capable of detecting thrombin activity.

15. A device for determining an anticoagulant in a blood sample comprising:
a) an analyzing unit comprising
(i) a first reaction zone providing a composition comprising
i) thrombin or a prothrombin activator converting prothrombin into thrombin that is FXa inhibitor insensitive, or mixtures thereof; and
ii) FXa or a prothrombin activator converting prothrombin into thrombin that is FXa inhibitor sensitive, or mixtures thereof;
wherein said first reaction zone is configured for contacting the composition with a blood sample suspected to comprise an anticoagulant for a time and under conditions which allow for thrombin activation in the blood sample;
(ii) a second reaction zone comprising a substrate capable of detecting thrombin activity, optionally an electrochemically detectable substrate; and
(iii) a detector which is capable of determining substrate turnover in the blood sample, thereby measuring thrombin activity; and
b) an evaluation unit which is capable of determining the anticoagulant based on the measured thrombin activity by comparison to a reference.

16. An analyte sensor for detecting an anticoagulant in a blood sample by electrochemical detection, comprising at least one working electrode according to claim 14, and at least an evaluation unit, which is capable of determining the anticoagulant based on the measured thrombin activity by comparison to a reference.

17. A kit for determining an anticoagulant in a blood sample comprising a composition and a substrate capable of detecting thrombin activity, wherein the composition comprises:
i) thrombin or a prothrombin activator converting prothrombin into thrombin that is FXa inhibitor insensitive, or mixtures thereof; and
ii) FXa or a prothrombin activator converting prothrombin into thrombin that is FXa inhibitor sensitive, or mixtures thereof; and
wherein the substrate is an electrochemically detectable substrate capable of detecting thrombin activity, optionally an electrochemically detectable peptide substrate.
